(19) United States
(12) Patent Application Publication
(10) Pub. No.: US 2025/0375429 A1
(43) Pub. Date: Dec. 11, 2025

(54) OBLIGATE PAIRING OF ORTHOSTERIC AND NON-ORTHOSTERIC GABA LIGANDS IN PHARMACEUTICAL FORMULATIONS

(71) Applicant: Smarter Not Harder, Inc., Rehoboth Beach, DE (US)

(72) Inventor: Theodore Borromeo ACHACOSO, Washington, DC (US)

(73) Assignee: Smarter Not Harder, Inc., Rehoboth Beach, DE (US)

(21) Appl. No.: 19/203,954

(22) Filed: May 9, 2025

Related U.S. Application Data
(60) Provisional application No. 63/653,215, filed on May 29, 2024.

(51) Int. Cl. A61K 31/437; A61K 31/165; A61K 31/197; A61K 31/42; A61K 31/455; A61K 45/06

(52) U.S. Cl. CPC A61K 31/437; A61K 31/165; A61K 31/197; A61K 31/42; A61K 31/455; A61K 45/06

(57) ABSTRACT

A pharmaceutical composition is provided and includes at least one orthosteric GABA receptor ligand that directly binds to at least one GABA-A receptor or at least one GABA-B receptor at the endogenous GABA binding site, and at least one non-orthosteric GABA receptor ligand that binds a site distinct from the endogenous GABA binding site on at least one GABA receptor type. This ensures the orthosteric and non-orthosteric ligands for each receptor type are co-present. The orthosteric and non-orthosteric ligands may be co-formulated for concurrent availability, such that the presence of the orthosteric ligand enables or enhances the therapeutic effect of the non-orthosteric ligand.

### RELATED APPLICATION

This application claims priority to U.S. Provisional Application 63/653,215 filed on May 29, 2024, the disclosures of which are incorporated by reference herein in their entireties.

### STATEMENT REGARDING FEDERALLY SPONSORED RESEARCH OR DEVELOPMENT

No federal or government funds were used in the development of this invention, and no rights are due to any government agency.

### FIELD OF THE INVENTION

The present invention relates to the field of pharmaceutical formulations and the treatment of GABA-related disorders and neuropharmacology, specifically to formulations that incorporate gamma-aminobutyric acid (GABA) receptor ligands in an obligate pairing of orthosteric and non-orthosteric binding-site GABA ligands.

### BACKGROUND OF THE INVENTION

Gamma-aminobutyric acid (GABA) is the principal inhibitory neurotransmitter in the mammalian central nervous system (CNS). It acts mainly via GABA-A and GABA-B receptors. GABA-A receptors are ionotropic and are ligand-gated chloride channels that respond to GABA by opening their respective chloride ion channels. GABA-B receptors are metabotropic and G-protein-coupled, that are coupled to calcium and potassium ion channels.

Orthosteric ligands bind the same site as the endogenous ligand, GABA, and may be agonists or antagonists. Non-orthosteric ligands, including allosteric ligands or modulators, bind other sites on the receptor, modifying its activity when orthosteric ligands are also bound. It has been standard practice to administer orthosteric and non-orthosteric ligands separately in treating various neurological disorders. However, each non-orthosteric GABA receptor modulator requires the concurrent presence of an orthosteric GABA ligand to function effectively.

GABA (gamma-aminobutyric acid) is well-recognized as the principal inhibitory neurotransmitter in the mammalian central nervous system. As such, many agents that either agonize GABA receptors or positively modulate receptor activity have been developed over the past several decades.

For example, several patents propose the use of GABA or GABAergic agonists for metabolic or autoimmune disorders rather than specifically for neurological applications. See, for example, U.S. Pat. No. 8,680,051 B2 which discloses a combination of a GABAergic agent with an incretin analog (such as GLP-1) to treat or prevent Type 1 diabetes; U.S. Pat. No. 9,463,174 B2 provides using GABA alongside a dipeptidyl peptidase-4 (DPP-4) inhibitor in a method for protecting B-cells and modulating autoimmunity. Other references provide single-agent GABA receptor modulators for CNS disorders. For example, U.S. Patent Application 2015/0313913 A1 discusses positive allosteric modulators (PAMs) of GABA-A receptors for autism spectrum disorder, focusing on subtype-selective PAMs; U.S. Pat. No. 8,344,138 B2 proposes positive allosteric modulators of the GABA-B receptor, indicating that such modulators can replicate or augment baclofen-like effects at reduced doses. EP 2621282 A2 discloses exogenous GABA for metabolic syndrome and Type 1 diabetes in combination with immunomodulatory strategies.

WO2019/055369 A1 (gaboxadol for narcolepsy), WO2012/064642 A1 (bamaluzole as a GABA-A agonist), and WO2015/095783 A1 (benzodiazepine-derived modulators for neurocognitive impairment) propose single-agent GABA agonist or modulator chemotypes. Despite the extensive variety of single-agent GABA receptor therapies, the art does not recognize the advantages of a pairing of an orthosteric agonist and non-orthosteric modulator in a unified composition for CNS disorders. The prior art neither teaches nor suggests co-formulating an orthosteric GABA receptor ligand with a dependent allosteric modulator in a single therapeutic composition. This leaves an unmet need for therapies that ensure an allosteric enhancer is always accompanied by an orthosteric activator.

For example, an allosteric agent like Valium® is often given alone, relying on endogenous GABA. However, oral GABA does not efficiently cross an intact blood-brain barrier, thus limiting its utility as a direct orthosteric agent in many formulations. Consequently, single-agent use can sometimes result in suboptimal outcomes, higher dose requirements, increased side effects, or reliance on inconsistent endogenous GABA release.

### SUMMARY OF THE INVENTION

This invention provides a novel therapeutic approach whereby at least one orthosteric GABA receptor agonist is formulated together with at least one non-orthosteric GABA receptor ligand, for example, a positive allosteric modulator (“PAM”) as an “obligate pair” in a single pharmaceutical composition. The orthosteric agonist (the so-called “key” to activate the receptor) is obligately paired with the non-orthosteric allosteric modulator (in essence the “boost” to enhance that activation), ensuring that the modulator's efficacy is realized by the concomitant presence of a GABA-activating agent. The co-formulated orthosteric and non-orthosteric ligands may be utilized for a wide variety of GABA-related neurological or psychiatric indications (for example, conditions such as anxiety, panic disorder, insomnia, epilepsy, muscle spasticity, and severe agitation or delirium). This approach-requiring the simultaneous presence of an orthosteric GABA ligand and an allosteric modulator-addresses an unmet need by ensuring receptor “priming” and consistent therapeutic action not taught or suggested by single-agent GABA receptor therapeutics.

The pharmaceutical composition of the present disclosure includes at least one orthosteric GABA receptor ligand that directly binds to at least one GABA-A receptor or at least one GABA-B receptor at the endogenous GABA binding site, and at least one non-orthosteric GABA receptor ligand that binds a site distinct from the endogenous GABA binding site on at least one GABA receptor type, thereby ensuring the orthosteric and non-orthosteric ligands for each receptor type are co-present, and optionally an electrolyte. The orthosteric and non-orthosteric ligands are co-formulated for concurrent availability, such that the presence of the orthosteric ligand enables or enhances the therapeutic effect of the non-orthosteric ligand.

### BRIEF DESCRIPTION OF THE DRAWINGS

FIG. 1 provides a schematic chart of a GABA-A receptor and illustrates, in schematic form, a GABA-A receptor with its distinct binding sites. The receptor is a pentamer (comprised of two α1, two β2, and one γ2 subunits, forming a central Cl⁻ channel pore). The diagram highlights the orthosteric GABA binding sites (labeled “GABA site” at the α1-β2 interfaces) and a representative non-orthosteric site (a benzodiazepine (BZD) site at an α1-γ2 interface).

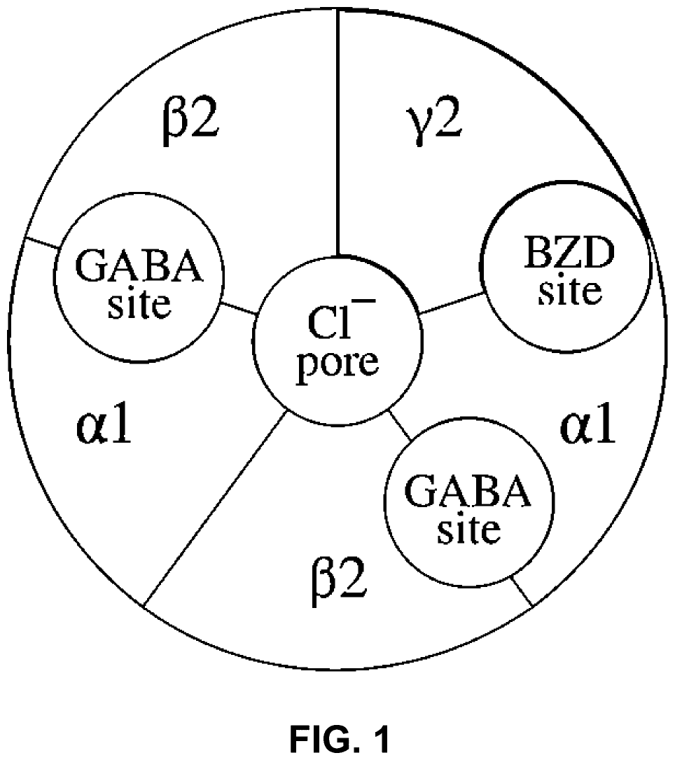

### DETAILED DESCRIPTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the compositions and methods of the present invention, is meant to indicate that the invention's listed elements are required, but that other elements which do not materially affect the basic and novel characteristics of the invention may also be present.

The pharmaceutical composition described herein obligates the paired presence of both an orthosteric GABA ligand and a non-orthosteric GABA ligand. By formulating at least one of each together, the issues of single-agent therapy (reliance on endogenous GABA for allosteric modulators, higher dosing needs, etc.) are overcome. When the two types of ligands are delivered concurrently, several advantages can be realized, including but not limited to:

Synergistic Efficacy: Orthosteric activation plus allosteric modulation can exceed the effect of either agent alone.

Dose-Sparing: Lower individual doses suffice when used in combination, reducing side effects.

Reliance Not Solely on Endogenous GABA: An exogenous agonist ensures receptor activation is not limited by physiological variations in GABA release.

Reduced Tolerance: Potentially slower tolerance buildup due to balanced dosing and multimodal receptor engagement.

Tailoring to Receptor Subtypes: Compositions may be designed to target GABA-A-mediated indications (e.g., anxiety, insomnia, epilepsy) or GABA-B-mediated indications (e.g., muscle spasticity such as that arising from multiple sclerosis or spinal cord injury, neuropathic pain), or both.

Indeed, as demonstrated in the exemplary scenarios described below, each obligate pair leverages these advantages in practice. For instance, the muscimol+honokiol combination (for insomnia) achieves the desired sedative effect with a lower muscimol dose, and the baclofen+3-OHB combination (for spasticity) produces the same muscle-relaxant outcome with less baclofen-illustrating the synergistic efficacy and dose-sparing benefits of the paired approach.

Orthosteric GABA ligands may bind directly to the primary active (orthosteric) site on GABA receptors. These may include endogenous and exogenous compounds that activate or block the receptor at the GABA site, as well as prodrugs that deliver GABA or GABA agonists. Examples of orthosteric GABA receptor ligands include GABA-A and GABA-B receptor agonists (for instance, GABA itself; muscimol; gaboxadol (THIP); baclofen; phenibut; as well as prodrugs like picamilon (N-nicotinoyl-GABA) that release GABA and progabide that release GABA analogs in the brain), and antagonists (e.g., for GABA-A: bicuculline, gabazine; for GABA-B: phaclofen, saclofen (2-hydroxy-saclofen), CGP55845, and SCH50911).

Non-orthosteric GABA ligands may bind to sites on the GABA receptor that are distinct from the GABA binding site, thereby modulating receptor activity indirectly. They may function as positive modulators (enhancing the receptor's response), negative modulators (diminishing the response), or other types of modulators (such as channel blockers or modulators acting at extracellular or intracellular sites). Examples of non-orthosteric ligands may include GABA-A receptor positive allosteric modulators (PAMs) such as kavalactones (e.g., kavain from Piper methysticum), honokiol and magnolol (from magnolia bark), apigenin, baicalin; valerenic acid (from valerian root), sage (Salvia officinalis) extract, dihydrohonokiol, benzodiazepines (e.g., diazepam, lorazepam), barbiturates (e.g., phenobarbital, pentobarbital), Z-drugs such as non-benzodiazepine hypnotic compounds (e.g., zolpidem, eszopiclone), neurosteroids (e.g., allopregnanolone and THDOC), ethanol, etomidate, propofol and bilobalide (from Ginkgo biloba); GABA-A receptor negative allosteric modulators (NAMs)/allosteric inverse agonists such as B-carbolines (e.g., DMCM, harmine, harmaline), Ro15-4513, and flumazenil (a benzodiazepine-site antagonist, often considered a neutral modulator); GABA-A receptor ion channel blockers such as picrotoxin (blocks the chloride channel of GABA-A receptors); GABA-A extracellular modulators such as zinc ions (Zn²⁺), pregnenolone sulfate, and thujone (from wormwood); GABA-A intracellular modulators: Protein kinase C (PKC), GABARAP, 14-3-3 proteins, and calmodulin; GABA-B receptor PAMs such as CGP7930, GS39783, BHFF and 3-OHB (3-hydroxybutanoic acid); and GABA-B intracellular binding partners (regulatory proteins) such as Gi/Go proteins, 14-3-3 proteins, GABABRIP1 (GABA-B-receptor interacting protein 1) and filamin A.

With respect to PAMs like honokiol and kavalactones, existing literature confirms PAMs need an orthosteric ligand present to exhibit their modulatory activity (e.g., benzodiazepines are inactive without GABA). Obligate pairing removes the uncertainty of endogenous GABA availability by supplying an exogenous agonist. Animal models of seizure, spasticity, and anxiety can test synergy and dose sparing, indicating that the combination can achieve therapeutic goals at lower individual doses.

Compounds like DMCM, harmine, harmaline, and other B-carbolines, as well as Ro15-4513, are benzodiazepine-site inverse agonists (negative allosteric modulators) acting on GABA-A receptors; however, they are not orthosteric ligands. In contrast, compounds such as phaclofen, saclofen (including 2-hydroxy-saclofen), CGP55845, and SCH50911 bind to the GABA-B receptor's orthosteric site as competitive antagonists and are therefore classified as orthosteric ligands.

To illustrate the invention's combination of at least one orthosteric GABA agonist with at least one non-orthosteric modulator so that the modulator's effect is always enabled by the presence of an agonist at the same receptor class (and its applicability to various GABA-related conditions), Table 1 below summarizes representative formulations, each featuring at least one orthosteric ligand (direct GABA-A or GABA-B agonist) and at least one non-orthosteric ligand (positive allosteric modulator). A brief rationale for each pairing explains how they synergize for prophylactic or therapeutic treatment benefits.

TABLE 1
Exemplary Orthosteric/Non-Orthosteric GABA Pairings

| | | Non- | | |
|---|---|---|---|---|
| | Orthosteric | Orthosteric | | |
| Indication | Ligand(s) | Ligand(s) | Formulation | Rationale |
| Anxiety | Picamilon | Kava | Oral capsule | Picamilon |
| (GABA-A) | (releases | kavalactones | or buccal | crosses the |
| | GABA in the | (positive | troche | blood-brain |
| | brain) | modulators) | | barrier |
| | | | | effectively, |
| | | | | delivering |
| | | | | exogenous |
| | | | | GABA, while |
| | | | | kavalactones |
| | | | | enhance GABA- |
| | | | | A receptor |
| | | | | activity. The |
| | | | | combination |
| | | | | provides |
| | | | | consistent |
| | | | | anxiolysis at |
| | | | | lower doses |
| | | | | than either |
| | | | | agent alone. |
| Insomnia | Muscimol | Honokiol or | Sublingual | Muscimol |
| (GABA-A) | (potent | dihydrohonokiol | lozenge or | strongly |
| | GABA-A | (PAM) | buccal troche | activates GABA- |
| | agonist) | | for rapid onset | A receptors, |
| | | | | inducing sleep |
| | | | | quickly. |
| | | | | Honokiol (or |
| | | | | dihydrohonokiol) |
| | | | | extends |
| | | | | inhibitory tone, |
| | | | | helping patients |
| | | | | remain asleep |
| | | | | longer and |
| | | | | reducing the |
| | | | | amount of |
| | | | | muscimol |
| | | | | needed. |
| Epilepsy | Phenibut | 3-OHB (positive | Oral | Phenibut raises |
| (GABA-B) | (central | modulator) | extended- | the seizure |
| | GABA-B | | release tablet | threshold via |
| | agonist) | | | GABA-B, while |
| | | | | 3-OHB |
| | | | | augments that |
| | | | | effect by binding |
| | | | | an allosteric site. |
| | | | | This synergy |
| | | | | supports |
| | | | | anticonvulsant |
| | | | | effects at lower |
| | | | | phenibut doses, |
| | | | | mitigating |
| | | | | sedation and |
| | | | | tolerance. |
| Muscle | Baclofen | CGP7930 or 3- | Oral or | Baclofen's direct |
| Spasticity | | OHB (PAM) | intrathecal | GABA-B action |
| (GABA-B) | | | infusion | alleviates |
| | | | | muscle spasms, |
| | | | | while CGP7930 |
| | | | | or 3-OHB |
| | | | | amplifies each |
| | | | | activation event. |
| | | | | This dose- |
| | | | | sparing strategy |
| | | | | lessens |
| | | | | baclofen's side |
| | | | | effects (e.g., |
| | | | | sedation, |
| | | | | muscle |
| | | | | weakness) and |
| | | | | may slow |
| | | | | tolerance. |
| Broad- | Muscimol | Midazolam | Intravenous | A multi-receptor |
| Spectrum | (GABA-A | (GABA-A PAM) + | (or | strategy for |
| Calming | agonist) + | 3-OHB | Intramuscular | severe agitation |
| (GABA-A + | Baclofen | (GABA-B PAM) | or | or delirium: |
| GABA-B) | (GABA-B | | Subcutaneous | muscimol/baclofen |
| | agonist) | | Injection) for | provide |
| | | | emergency | orthosteric |
| | | | sedation | activation, while |
| | | | | midazolam/3- |
| | | | | OHB further |
| | | | | enhance |
| | | | | inhibition at both |
| | | | | receptor types. |
| | | | | Requires careful |
| | | | | monitoring due |
| | | | | to strong |
| | | | | sedative effects. |

### Exemplary Explanations (for Pairings in Table 1):

Anxiety (GABA-A): Rationale: Because endogenous GABA levels can fluctuate and GABA itself crosses the blood-brain barrier poorly, providing an exogenous source (via picamilon, which releases GABA in the brain) ensures there is always an agonist at the receptor. The kavalactones then allosterically potentiate this GABA-A-mediated inhibitory tone, reducing anxiety without reliance on high doses of either agent alone.

Insomnia (GABA-A): Rationale: Muscimol directly gates the receptor for swift sleep induction, while honokiol or dihydrohonokiol reduces the risk of abrupt awakenings by prolonging receptor activation. This synergy helps avoid higher doses of muscimol, which could otherwise cause unwanted psychoactive effects (e.g., hallucinations).

Epilepsy (GABA-B): Rationale: Phenibut's GABA-B agonism stabilizes neuronal excitability. 3-OHB (a GABA-B receptor positive allosteric modulator) modulates these receptors allosterically, enhancing each activation event. By combining the two, total phenibut exposure can be lowered, potentially reducing side effects or tolerance over long-term treatment.

Muscle Spasticity (GABA-B): Rationale: Baclofen is well-established for treating spasticity but can cause sedation and tolerance at higher doses. Adding CGP7930 or 3-OHB as a positive modulator allows a lower dose of baclofen to achieve the same muscle-relaxant effect, improving patient comfort and functionality.

Broad-Spectrum Calming (GABA-A+GABA-B): Rationale: For extreme agitation or delirium, addressing both receptor subtypes (GABA-A and GABA-B) simultaneously can deliver rapid tranquilization. For example, an orthosteric GABA-A agonist (e.g., muscimol) paired with an orthosteric GABA-B agonist (e.g., baclofen) can be co-administered along with allosteric enhancers for each receptor (midazolam, a benzodiazepine GABA-A PAM, and 3-OHB, a GABA-B PAM), thereby engaging both inhibitory pathways for a maximal combined effect. Since both GABA-A and GABA-B are engaged orthosterically and allosterically, sedation is profound-thus restricting use to controlled clinical settings.

Each scenario demonstrates the obligate pairing of an agonist with a compatible modulator to ensure the receptor is always “primed” for allosteric enhancement.

Various electrolytes such as potassium chloride, sodium chloride, sodium bicarbonate, magnesium chloride, calcium chloride, potassium phosphate, sodium citrate, and magnesium sulfate may be included with the pharmaceutical composition.

The pharmaceutical composition may be delivered as a single dose, concurrently or in a controlled or sustained release profile designed to overlap in time in vivo. The pharmaceutical composition may be delivered transdermally, orally, transmucosally, rectally, vaginally, sublingually, buccally, by inhalation, by injection, via a patch, via nanoparticle delivery, or by nebulizer, the selection of which will be within the skill of one in the art. Oral administration of the pharmaceutical composition may include formulations in the form of a paste, resin, oil, powder or liquid in tablet, capsule, troche, lozenge, gummy, and film form.

The pharmaceutical composition/formulation may include pharmaceutically acceptable additives such as suspending agents, preservatives, colorants, sweeteners, binders, and disintegrants.

In one embodiment, additional pharmaceuticals, nutraceuticals, dietary supplements, or homeopathic compositions may be included. The additional formulations may be blended with the pharmaceutical composition or may be co-administered. The composition may further comprise one or more additional therapeutic agents such as therapeutic FDA-approved APIs (e.g., NSAIDs such as ibuprofen, naproxen, diclofenac; COX-2 inhibitors; acetaminophen; antihistamines; SSRIs; SNRIs; B-blockers; cannabinoids) formulated either in the same dosage unit or as a coordinated multi-unit therapy. Exemplary nutraceuticals may be derived from herbal materials and may be in the form of a herbal extract. Exemplary herbal materials may include, but are not limited to, one or more of: echinacea, St. John's wort, valerian root, milk thistle seed, Siberian ginseng, nettle leaf, holy basil, ginkgo, gotu kola, astragalus, goldenseal, dong quai, ginseng, bilberry, green tea, hawthorne, ginger, turmeric, Rhodiola rosea, black cohosh, cat's claw, chamomile, dandelion, chaste tree berry, feverfew, garlic, horse chestnut, licorice, eyebright, yohimbe, poppy, black pepper, arnica, black elderberry, ashwagandha, acacia, hemp (including cannabinoids, for example, CBD, CBG, and CBN), acai, allspice, skullcap, burdock, and maca, and various other herbal materials listed, for example, in the Materia Medica of Western Herbs by C. Fisher or Homoeopathic Materia Medica by H. Boericke.

In certain embodiments, the invention provides a kit that includes at least one orthosteric GABA receptor ligand and at least one non-orthosteric GABA receptor ligand packaged in separate containers or compartments. The kit may further comprise instructions for combining or co-administering these ligands so that the orthosteric agent is present at the receptor when the allosteric modulator exerts its effect. For example, for anxiety, an orthosteric GABA-A agonist (e.g., a muscimol buccal troche) can be provided alongside a positive allosteric modulator (e.g., a kavalactone such as kavain in a buccal troche), with dosing guidelines (e.g., quartering or halving each troche to titrate the dose) ensuring their optimally titrated dose and optimal concurrent delivery. By requiring the paired use of both agents, the kit guarantees the synergy and dose-sparing benefits described above. The kits may also include the added pharmaceuticals, nutraceuticals, dietary supplements, or homeopathic compositions described above. Kits may also include additional excipients, measuring devices, or written dosage instructions, all aimed at preserving the obligate pairing concept.

Unless otherwise stated, all examples and embodiments herein are illustrative and do not limit the scope of the claims. Various changes, modifications, and alterations may be made without departing from the spirit and scope of the invention.

That which is claimed is:

1. A pharmaceutical composition comprising:
(a) at least one orthosteric GABA receptor ligand that directly binds to at least one GABA-A receptor or at least one GABA-B receptor at the endogenous GABA binding site; and
(b) at least one non-orthosteric GABA receptor ligand that binds a site distinct from the endogenous GABA binding site on at least one GABA receptor type, thereby ensuring the orthosteric and non-orthosteric ligands for each receptor type are co-present; wherein the orthosteric and non-orthosteric ligands are co-formulated for concurrent availability, such that the presence of the orthosteric ligand enables or enhances the therapeutic effect of the non-orthosteric ligand.

2. The pharmaceutical composition of claim 1, further comprising an electrolyte.

3. The pharmaceutical composition of claim 1, wherein the orthosteric ligand is a GABA-A or GABA-B receptor agonist.

4. The pharmaceutical composition of claim 3, wherein the GABA-A or GABA-B ligand is selected from the group consisting of gamma-aminobutyric acid (GABA), muscimol, gaboxadol, baclofen, phenibut, progabide, picamilon, and salts or prodrugs thereof.

5. The pharmaceutical composition of claim 1, wherein the non-orthosteric ligand is selected from the group consisting of benzodiazepines, barbiturates, non-benzodiazepine hypnotic compounds, neurosteroids, ethanol, kava kavalactones, honokiol, valerian extracts, 3-hydroxybutyric acid (3-OHB), CGP7930, GS39783, and pharmaceutically acceptable analogs or derivatives thereof.

6. The pharmaceutical composition of claim 1, wherein the composition is formulated for at least one route of administration selected from the group consisting of transdermally, orally, transmucosally, rectally, vaginally, sublingually, buccally, by inhalation, by injection, via a patch, via nanoparticle delivery, or by nebulizer.

7. The pharmaceutical composition of claim 1, wherein the composition is presented as a single dosage unit that delivers one or more of the orthosteric and one or more of the non-orthosteric ligands concurrently or in controlled-release profiles designed to overlap in time in vivo.

8. The pharmaceutical composition of claim 1, wherein administering the composition results in a synergistic therapeutic effect, allowing a lower dose of at least one of the ligands compared to monotherapy with that ligand.

9. The pharmaceutical composition of claim 1, wherein the orthosteric ligand and the non-orthosteric ligand both target GABA-A receptors, thereby enhancing GABA-A-mediated inhibitory tone.

10. The pharmaceutical composition of claim 1, wherein the orthosteric ligand and the non-orthosteric ligand both target GABA-B receptors, thereby enhancing GABA-B-mediated inhibitory tone.

11. The pharmaceutical composition of claim 1, further comprising one or more pharmaceutically acceptable excipients, carriers, or stabilizers that maintain the chemical stability and bioavailability of both ligands.

12. The pharmaceutical composition of claim 1, further comprising at least one therapeutic agent selected from the group consisting of FDA-approved APIs, nutraceuticals, dietary supplements and homeopathic compositions.

13. A method of treating a GABA-related disorder in a subject in need thereof, the method comprising administering to the subject a therapeutically effective amount of the pharmaceutical composition of claim 1, such that the orthosteric ligand and the non-orthosteric ligand are delivered concurrently.

14. The method of claim 13, wherein the GABA-related disorder is selected from the group consisting of anxiety, panic disorder, insomnia, epilepsy, acute agitation, severe anxiety, or delirium requiring rapid sedation.

15. The method of claim 13, wherein the GABA-related disorder is muscle spasticity associated with multiple sclerosis, spinal cord injury, or neuropathic pain syndromes.

16. The method of claim 13, wherein the composition is administered transdermally, orally, transmucosally, rectally, vaginally, sublingually, buccally, by inhalation, by injection, via a patch, via nanoparticle delivery, or by nebulizer, under conditions that ensure simultaneous bioavailability of the orthosteric and non-orthosteric ligands at the receptor site.

17. A kit comprising:
(a) the pharmaceutical composition of claim 1; and
(b) instructions for use describing administration of said composition to treat a condition that benefits from enhanced therapeutic effect of the non-orthosteric ligand.

18. The kit of claim 17, wherein the instructions specify at least one dosage regimen for an indication selected from anxiety, insomnia, muscle spasticity, epilepsy, severe agitation or delirium, and further detail concurrent or sequential administration guidelines designed to preserve the obligate pairing of the orthosteric and non-orthosteric ligands.

19. The kit of claim 17, wherein the pharmaceutical composition is provided in separate compartments for each active ingredient, the contents of which are combined prior to administration to ensure concurrent delivery, wherein the kit provides for titratable dosing of both ligands.

20. The kit of claim 17, wherein the kit further includes at least one therapeutic agent selected from the group consisting of FDA-approved APIs, nutraceuticals, dietary supplements and homeopathic compositions.

21. A method of formulating a pharmaceutical composition for providing an obligate pairing, the method comprising:
(a) selecting at least one orthosteric GABA receptor ligand as an active ingredient;
(b) selecting at least one non-orthosteric GABA receptor ligand as an active ingredient; and
(c) combining the selected ligands in a single pharmaceutical formulation such that a non-orthosteric ligand's modulatory effect is enabled or enhanced by the simultaneous presence of an orthosteric ligand.

22. The method of claim 21, further comprising incorporating one or more excipients that stabilize each active ingredient, and adjusting release profiles so both ligands overlap in vivo.

23. The method of claim 21, wherein the formulation is prepared for an administration route selected from the group consisting of transdermally, orally, transmucosally, rectally, vaginally, sublingually, buccally, by inhalation, by injection, via a patch, via nanoparticle delivery, or by nebulizer, and includes processing steps that ensure appropriate solubility or bioavailability of each ligand under that route.
